(19) United States
(12) Patent Application Publication
(10) Pub. No.: US 2023/0277832 A1
(43) Pub. Date: Sep. 7, 2023

(54) Peelable Cathlock

(71) Applicant: Bard Peripheral Vascular, Inc., Franklin Lakes, NJ (US)

(72) Inventor: Niki Hale, Bountiful, UT (US)

(21) Appl. No.: 18/018,529

(22) PCT Filed: Jul. 13, 2021
(86) PCT No.: PCT/US21/41467

§ 371 Date: Jan. 27, 2023

Related U.S. Application Data
(60) Provisional application No. 63/057,704, filed on Jul. 28, 2020.

(51) Int. Cl. A61M 39/02

(52) U.S. Cl. CPC A61M 39/0247

(57) ABSTRACT

A peelable cathlock device is configured to allow a catheter or a port to be able to be replaced independently of each other. The peelable cathlock includes a body defining a cylindrical shape and extending from a first end to a second end. The body defines a lumen providing fluid communication between the first end, which is configured to attach to the proximal end of a catheter, and the second end configured to attach to a port. The peelable cathlock includes a tear line extending along a portion of the body are configured to allow the body to separate along the tear line when a force is applied.

### PRIORITY

This application claims the benefit of priority to U.S. Provisional Application No. 63/057,704, filed Jul. 28, 2020, which is incorporated in its entirety into this application.

### BACKGROUND

Catheter systems can include a catheter locking device (hereinafter, “cathlock”), which can fluidly couple various vascular access devices within the system, such as a catheter to a vascular access port. The cathlock can be selectively detachable to allow one of the catheter or the port to be exchanged independently of each other. For example, the catheter system can be placed subcutaneously in a chest area of a patient. Occasionally, the catheter portion of the system requires replacing, such as to clear a blockage. The catheter can be replaced in a procedure known as a catheter exchange. Some catheter systems do not allow the catheter to be detachable from the port, requiring the catheter and the port to be replaced simultaneously. This can be expensive, time consuming and detrimental to the patient. Disclosed herein are embodiments of a cathlock device, and method of use, that allow the catheter or the port to be replaced independently of each other.

### SUMMARY

Disclosed herein is a peelable cathlock including, a body defining a cylindrical shape and extending from a first end to a second end, the body defining a lumen providing fluid communication between the first end and the second end, the first end is configured to attach to a proximal end of a catheter, and the second end is configured to attach to a port stem, and a tear line extending along a portion of the body and configured to allow the body to separate therealong when a force is applied.

In some embodiments, the tear line includes one of a score line, groove, laser cut line, perforation, a thinned portion of a wall of the body. In some embodiments, the peelable cathlock further includes, a frangible band extending along a portion of the body and defined by a first tear line and a second tear line. In some embodiments, the peelable cathlock further includes a pull tab coupled to one of the body or the frangible band, proximate one of the first end or the second end, and configured to allow a user to grasp the pull tab and urge the pull tab away from the body to separate the body along one of the first tear line or the second tear line. The frangible band extends in a helical shape about a longitudinal axis of the body.

In some embodiments, a first portion of the helical frangible band is disposed adjacent a second portion of the helical frangible band to form a portion of the body. The frangible band extends parallel to a longitudinal axis of the body between the first end and the second end. The first tear line extends from the first end to the second end and the second tear line extends along a portion of the body from one of the first end or the second end. The frangible band extends along a portion of the body at an angle relative to longitudinal axis. The cathlock includes one of a plastic, polymer, elastomer, polytetrafluoroethylene (PTFE), metal, or alloy material.

Also disclosed is a method of removing a cathlock from a catheter system including, providing a cathlock including a body defining a substantially cylindrical shape and extending from a first end to a second end, and a tear line extending along a portion of the body, the cathlock providing fluid communication between a port and a catheter, grasping a portion of the cathlock, urging the portion of the cathlock away from the catheter system, and separating cathlock along the tear line.

In some embodiments, the tear line includes one of a score line, groove, laser cut line, perforation, or a thinned portion of a wall of the body. In some embodiments, the method further includes a frangible band extending along a portion of the body and defined by a first tear line and a second tear line. Grasping a portion of the cathlock further includes grasping a pull tab coupled to one of the body or the frangible band, proximate one of the first end or the second end. The frangible band extends in a helical shape about a longitudinal axis of the body. A first portion of the helical frangible band is disposed adjacent a second portion of the helical frangible band to form a portion of the body. The frangible band extends parallel to a longitudinal axis of the body between the first end and the second end.

In some embodiments, the first tear line extends from the first end to the second end and the second tear line extends along a portion of the body from one of the first end or the second end. The frangible band extends along a portion of the body at an angle relative to longitudinal axis. The cathlock includes one of a plastic, polymer, elastomer, polytetrafluoroethylene (PTFE), metal, or alloy material.

These and other features of the concepts provided herein will become more apparent to those of skill in the art in view of the accompanying drawings and following description, which describe particular embodiments of such concepts in greater detail.

### DRAWINGS

FIG. 1 illustrates a plan view of a peelable cathlock providing fluid communication between a port and a catheter, in accordance with some embodiments.

FIGS. 2A-2H illustrate plan views of various embodiments of peelable cathlocks, in accordance with some embodiments.

FIGS. 3A-3B illustrate a cross sectional view of a peelable cathlock including a tear line, in accordance with some embodiments.

FIGS. 4A-4B illustrate an exemplary method removing a peelable cathlock, in accordance with some embodiments.

FIGS. 5A-5D illustrate an exemplary method of removing a peelable cathlock from a catheter system, in accordance with some embodiments.

FIG. 6 illustrates a block diagram of an exemplary method of removing a peelable cathlock from a catheter system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

As used herein, the term “tear line” includes a line of weakness configured to allow separate therealong and can include a score line, groove, laser cut line, perforation, a thinned portion, or similar structure to facilitate separation therealong. Unless defined otherwise, all technical and scientific terms used herein have the same meaning as commonly understood by those of ordinary skill in the art.

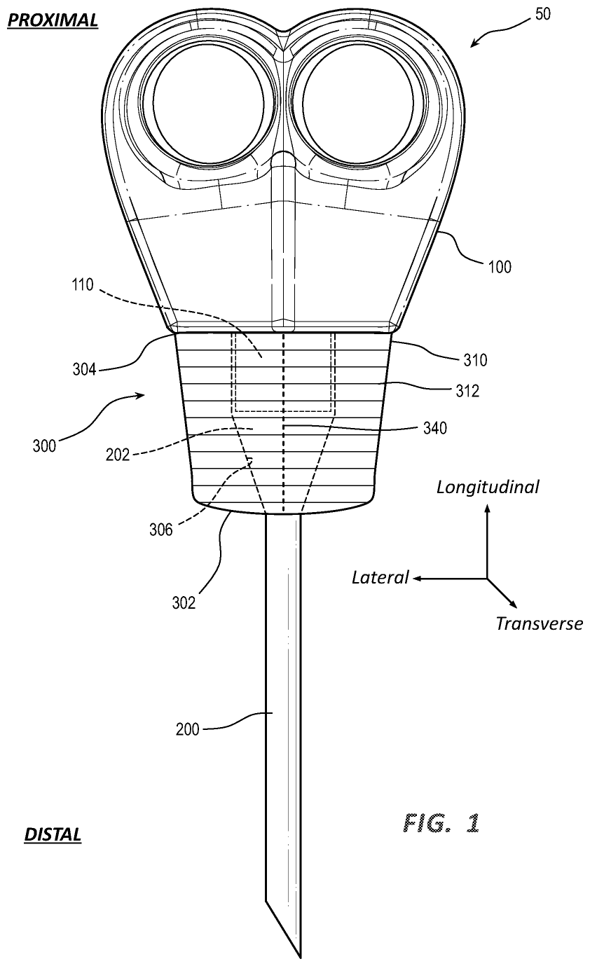

FIG. 1 illustrates a plan view of a catheter system 50 including a port 100, a catheter 200 and a peelable cathlock (“cathlock”) 300 configured to connect the catheter 200 to the port 100 and provide fluid communication therebetween. In some embodiments, the peelable cathlock 300 includes a cathlock body (“body”) 310 defining a substantially cylindrical shape and extending from a first end 302, disposed proximate the catheter 200, to a second end 304, disposed proximate the port 100. The body 310 can define a lumen 306 extending from the first end 302 to the second end 304. The first end 302 can be configured to attach to the catheter 200, and the second end 304 can be configured to attach to the port 100. In some embodiments, the body 310 includes a tear line 340 configured to allow the body 310 to separate therealong, as discussed in more detail herein.

In an embodiment, the proximal end 202 of the catheter 200 can engage the port stem 110 to provide fluid communication between one or more lumens of the port stem 110 and one or more lumens of the catheter 200. It should be appreciated that although the port 100 is shown with two access openings, a port with a single access opening or more than two access openings could be used with the inventive cathlock 300. The present invention is not limited by the configuration of the port 100 or the catheter 200 configured to connect to the port 100 as it can be configured to connect any port and catheter combination. Following engagement of the catheter 200 and port stem 110, the cathlock 300 is disposed over both to secure the catheter 200 to the port 100 and inhibit disengagement of the catheter 200 from the port 100, or inhibit leakage.

In an alternate embodiment, the cathlock 300 can define one or more lumen 306, each cathlock lumen 306 configured to provide fluid communication between a lumen of the port stem 110 and a lumen of the catheter 200. The port stem 110 can engage a second end 304 of the cathlock 300 to provide fluid communication between a lumen of the port stem 110 and a lumen 306 of the cathlock 300. Further, the catheter 200 can engage a first end 302 of the cathlock 300 to provide fluid communication between a lumen 306 of the cathlock 300 and a lumen of the catheter 200.

The peelable cathlock 300 can include a tear line 340 extending along a portion of the body 310. The tear line 340 can provide a line of weakness configured to allow the body 310 to separate along an axis when a force is applied thereto. As shown in FIG. 1, in an embodiment, the axis of the tear line 340 can extend longitudinally along the body 310 between a first end 302 and a second end 304. In an embodiment, the axis of the tear line 340 can extend helically about the body 310. However, other configurations are contemplated, as described herein. In an embodiment, the tear line 340 can include score line, groove, laser cut line, perforation, a thinned portion, or similar structure to facilitate separation therealong. It is important to note that in an intact configuration, the tear line 340 still provides a fluid tight seal to prevent any fluid, disposed within the cathlock lumen 306 from passing through the first tear line 340A and second tear line 340B.

In some embodiments, the catheter system 50, including the cathlock 300, the catheter 200, and the port 100, can be formed integrally during the manufacturing process. In other embodiments, the cathlock 300, catheter 200 and port 100 can be manufactured separately and assembled before placement within a patient. In an embodiment, one or more of the cathlock 300, catheter 200 or port 100 may be coupled to a catheter system 50 that is already disposed within the patient. In some embodiments, the catheter system 50 can be assembled by urging a proximal end 202 of the catheter 200 into the first end 302 of the cathlock body 310. In some embodiments, the catheter system 50 can be assembled by urging a port stem 110 of a port 100 into the second end 304 of the cathlock body 310. One of the catheter proximal end 202 or the port stem 110 can engage the cathlock 300 in an interference fit, press fit, snap fit, protrusion and recess, pinion and detent, barbed engagement, combinations thereof, or the like. In an embodiment, one of the catheter proximal end 202 or the port stem 110 can be coupled to the cathlock 300 by adhesive, ultrasonic welding, bonding, combinations thereof, or the like. In some embodiments, the cathlock 300 may be 3D printed or injection molded. In some embodiments, the cathlock 300 may be formed of a plastic, polymer, elastomer, polytetrafluoroethylene (PTFE), metal, alloy, combinations thereof, or the like.

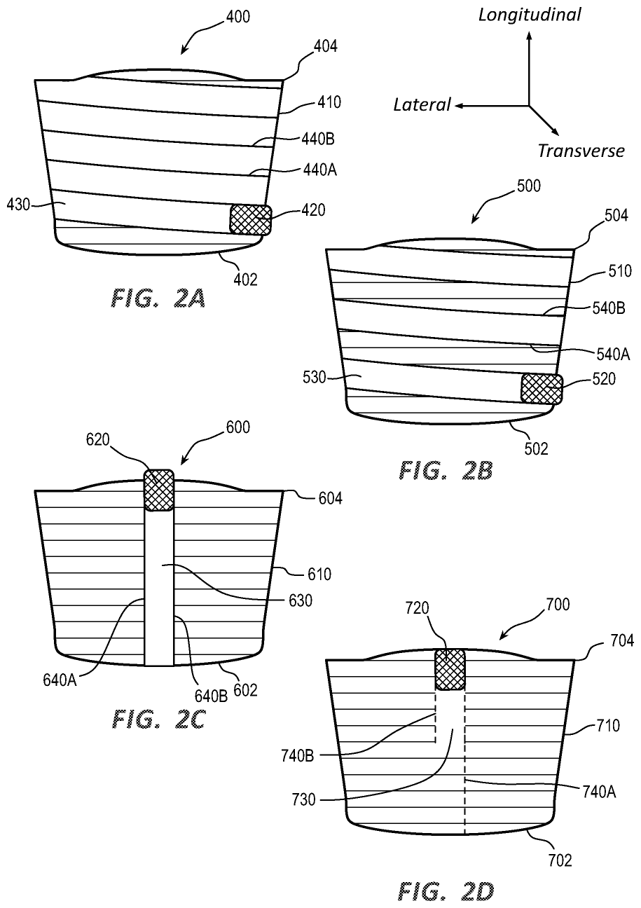

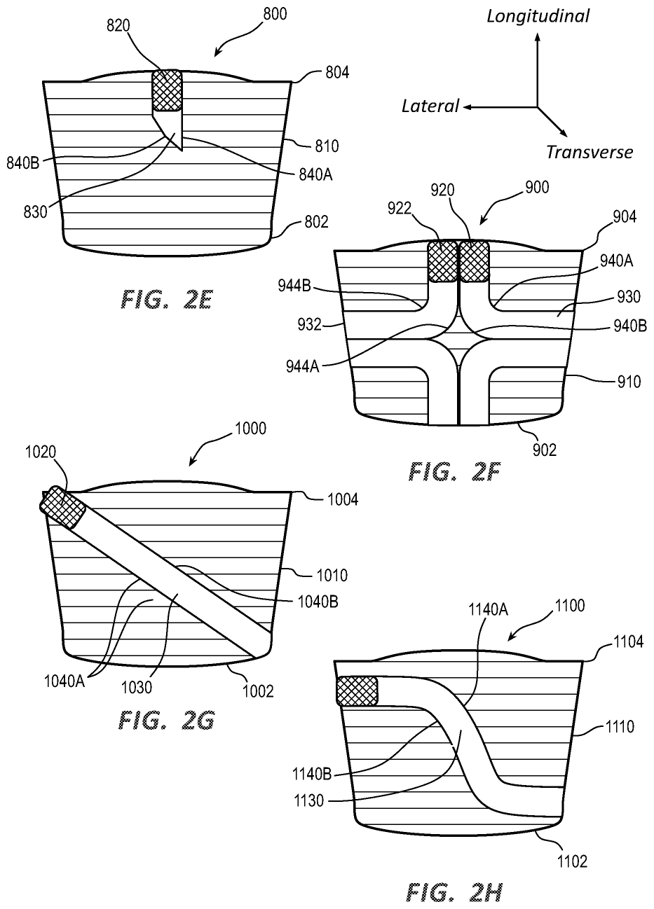

In some embodiments, as illustrated in FIGS. 2A-2H, the tear line 340 may include more than one tear lines that can be configured in a variety of ways to facilitate easier separation of the cathlock 300 into at least one piece. For example, in an embodiment as illustrated in FIG. 2A, the cathlock body 410 can include a pull tab 420 configured to facilitate grasping the body 410 and separation of the body 410 along a tear line 440, e.g. a first tear line 440A and a second tear line 440B. In an embodiment, the pull tab 420 is coupled to a portion of the body 410. In an embodiment, the pull tab 420 is coupled to a frangible band 430, which is defined by the first tear line 440A and the second tear line 440B. The frangible band 430 is configured to separate from the body 410 of the cathlock 400 along the first tear line 440A and the second tear line 440B. In an embodiment, adjacent portions of the frangible band 430 can be coupled together in a helical shape to define the cathlock body 410. A force can be applied to the pull tab 420, which is coupled to the frangible band 430 at a first end 402 of the cathlock body 410, and can unwrap the frangible band 430 in a helical manner. This allows the cathlock 400 to be separated from the catheter 200 and the port 100. In an embodiment, the pull tab 420 can be coupled to the frangible band 430 at a second end 404 of the cathlock body 410.

In some embodiments, the pull tab 420 can be substantially rectangular shaped. However, it will be appreciated that the pull tab 420 can be of various shapes such as oval, square, trapezoid, or the like. In some embodiments, the pull tab 420 can be textured to provide improved frictional interaction with a grasping medical device, such as forceps, hemostats, or the like. In some embodiments, the pull tab 420 can be coupled to a portion of the peelable cathlock 400 (e.g. FIG. 2D) and can facilitate separation of the cathlock 400 along the first tear line 440A, the second tear line 440B or both. In embodiments, the pull tab 420 can be disposed proximate a first end 402, proximate a second end 404, or at a point disposed therebetween.

As shown in FIG. 2B, in an embodiment, the frangible band 530 of the cathlock 500 can wrap around the cathlock body 510 in a spiral, or helical configuration. In this configuration, adjacent portions of the helical frangible band 530 can include portions of the catheter body 510 interposed therebetween. The frangible band 510, defined by the first tear line 540A and the second tear line 540B can be separated from the cathlock body 510 by applying a force on the pull tab 520 which is coupled to the frangible band 530 at a first end 502 of the cathlock body 510. The frangible band 530 can separate from the cathlock body 510, allowing the cathlock 500 to be detached from one of the catheter 200 or the port 100. In some embodiments, the pull tab 520 can be coupled to the frangible band 530 at the second end 504 of the cathlock body 510.

FIG. 2C illustrates an embodiment, including a frangible band 630, defined by a first tear line 640A and a second tear line 640B, extending substantially parallel to a longitudinal axis. One of the frangible band 630, first tear line 640A or second tear line 640B can extend from the second end 604 of the cathlock body 610 to the first end 602 of the body 610 with the pull tab 620 coupled to the second end 604 of frangible band 630. In this configuration, a force applied to the pull tab 620 can separate the frangible band 630 from the body 610 at a second end 604, and can continue to separate along one of the tear lines 640A, 640B moving towards the first end 602. This can separate the cathlock 600 into the frangible band 630 and the cathlock body 610. In some embodiments, the pull tab 620 may be coupled to the first end of the frangible band 630 and the separation may start at the first end and move towards the second end 602.

FIG. 2D illustrates and embodiment, including a cathlock 700 with a shortened frangible band 730. The cathlock 700 can include a pull tab 720 coupled to a second end 704 of the frangible band 730, as defined by a first tear line 740A and a second tear line 740B. The first tear line 740A can extend from the second end 704 to the first end 702. The second tear line 740B can extend from the second end 704 to a point disposed between the second end 704 and the first end 702. As such, a portion of the frangible band 730 can remain connected to the cathlock body 710, even after the first tear line 740A and second tear line 740B are separated. Advantageously, the frangible band 730 and the cathlock body 710 can be removed as one piece.

FIG. 2E illustrates a cathlock 800 with a shortened frangible band 830. In this embodiment, the cathlock 800 includes a pull tab 820 coupled to a second end 804 of the frangible band 830, as defined by a first tear line 840A and a second tear line 840B. In an embodiment, the second tear line 840B intersects the first tear line 840A at a point disposed between the first end 802 and the second end 804, to define a distal end of the frangible band 830. This allows for the frangible band 830 to separate from the cathlock body 810 after the first tear line 840A and second tear line 840B are separated. Further this allows one end of the cathlock 800, e.g. the second end 804 to separate from the port 100 while the opposite end, e.g. the first end 802 remains attached to the catheter 200. Advantageously, this allows the cathlock body 810 to be removed along with the catheter 200. In an embodiment, the frangible band 830 can extend from the first end 802 of the body 810 and allow the cathlock 800 to separate from catheter 200 while remaining attached to the port 100.

In some embodiments, the cathlock may include more than one frangible band and more than one pull tab. FIG. 2F illustrates an embodiment of a cathlock 900 that includes a first pull tab 920 coupled to a first frangible band 930 and a second pull tab 922 coupled to a second frangible band 932, each at second end 904 of the cathlock body 910. The first frangible band 930 is defined by a first tear line 940A and a second tear line 940B. The second frangible band 932 is defined by a third tear line 944A and a fourth tear line 944B. In this embodiment, the first frangible band 930 and the second frangible band 932 wrap around the cathlock body 910 in a mirrored configuration. In some embodiments, the first pull tab 920 can be coupled to the first frangible band 930 and the second pull tab 922 can be coupled to the second frangible band 932 at the first end 902 of the cathlock body 910. In some embodiments, the first pull tab 920 can be coupled to the first frangible band 930 at the first end 902 or the second end 904, and the second pull tab 922 can be coupled to the second frangible band 932 at the second end 904 or the first end 902 of the cathlock body 910. In the embodiment illustrated in FIG. 2F, a force applied to one of the first pull tab 920, or the second pull tab 922, either simultaneously or sequentially, can separate the cathlock body 910 into two or more pieces.

FIG. 2G illustrates a cathlock 1000 that includes a frangible band 1030, a first tear line 1040A, and a second tear line 1040B, extending at an angle relative to the longitudinal axis. The cathlock 1000 can further include a pull tab 1020 coupled to the frangible band 1030 at the second end 1004 of the cathlock body 1010 proximate a first lateral side. A distal end of the frangible band 1030 can be disposed proximate the first end 1002, and proximate a second lateral side, opposite the first lateral side. In some embodiments, the pull tab 1020 can be coupled to the frangible band 1030 at the first end 1002 of the cathlock body 1010. FIG. 2H illustrates an embodiment of a cathlock 1100 wherein the frangible band 1130, extends in a non-linear shape along the cathlock body 1110, with a pull tab 1120 coupled to the frangible band 1130 at the first end 1102. In some embodiments, the pull tab 1120 may be coupled to the second end 1104 of the cathlock body 1110. In an embodiment, the first tear line 1140A and the second tear line 1140B can extend parallel to each other along either a linear path or a non-linear path. In an embodiment, the first tear line 1140A and the second tear line 1140B can extend along non-parallel, linear or non-linear paths, relative to each other.

The configurations of the tear lines and frangible bands in FIGS. 2A-2H are only exemplary and various configurations are contemplated. Furthermore, configurations of tear lines and frangible bands in FIGS. 2A-2H are not to be taken as limiting in any way.

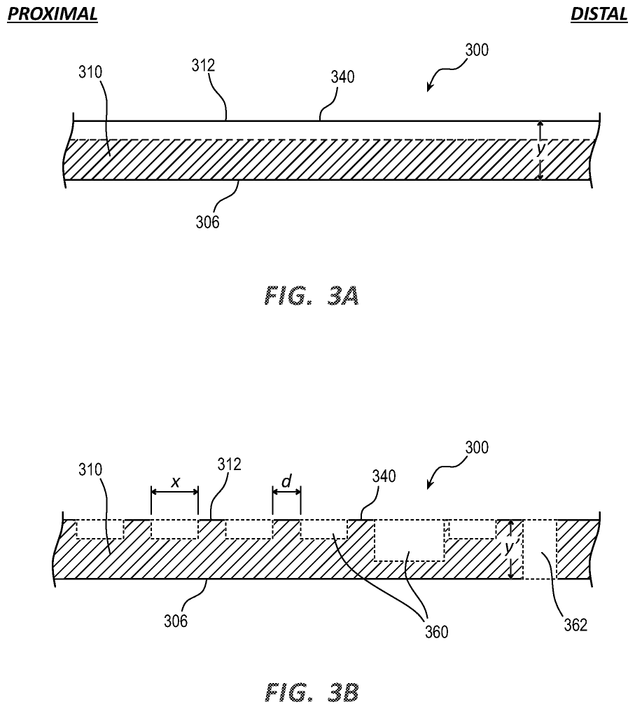

FIGS. 3A-3B illustrates a cross section of the cathlock body 310 of FIG. 1 including a tear line 340. In an embodiment, the tear line 340 can include a groove, or thinned portion extending partially through a wall thickness (y) of the catheter body 310. As shown in FIG. 3A, in an embodiment the thinned portion of the tear line 340 can extend continuously from a proximal end to a distal end. As shown in FIG. 3B, in some embodiments, the tear line 340 includes a plurality of indentations 360 extending partially through a wall thickness (y) of the cathlock body 310. The plurality of indentations 360 can extend along a portion of the tear line 340. One or more indentations of the plurality of indentations 360 can extend at the same length (x) or at different lengths (x). Similarly, one or more indentations of the plurality of indentations 360 can be spaced at the same distance (d) or at different distances (c). In an embodiment, the tear line 340 may include a perforation 362 that extends entirely through a wall thickness (y) of the catheter body 310. In an embodiment, the tear line 340 may include indentation 360 that extend at different depths through the wall thickness (y). It is important to note that while the tear line 340 provides a line of weakness, the tear line 340 does not compromise the integrity of the fluid seal of the cathlock 300. This maintains fluid communication through the lumen 306 of the cathlock 300 between the port 100 and the catheter 200.

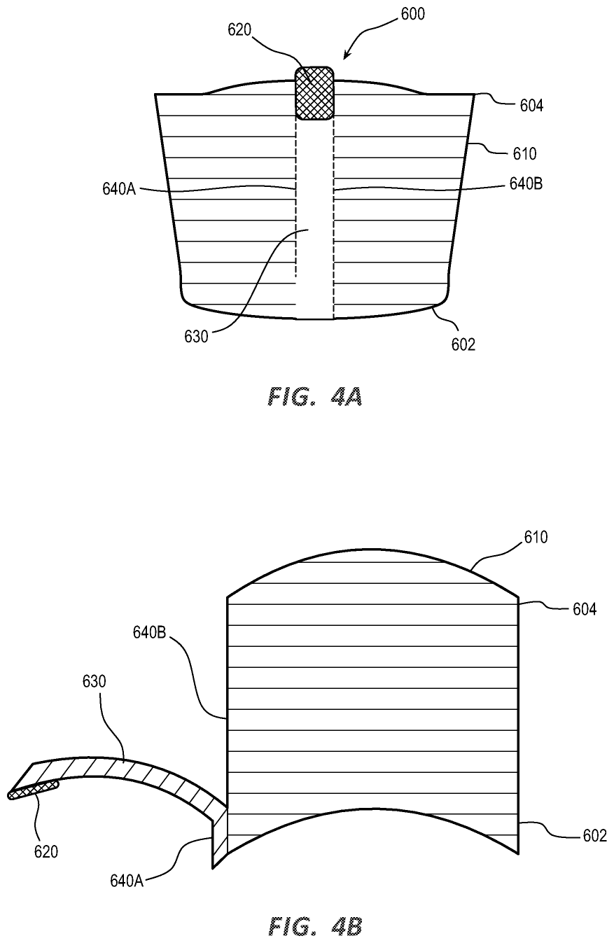

FIGS. 4A-4B illustrate an exemplary method removing a peelable cathlock, in accordance with some embodiments. The cathlock 600 includes a body 610 including a first end 602 and a second end 604. The cathlock 600 further includes a pull tab 620 coupled at the second end 604 to a frangible band 630. In an embodiment, one of the first tear line 640A or the second tear line 640B can extend partially along a portion of the body 610 allowing a portion of the frangible band 630 to remain attached to the body 610. For example, as illustrated in FIG. 4B, a first tear line 640A extends partially along a portion of the body 610 and a second tear line 640B extends from the first end 602 to a second end 604. As a force is applied to the pull tab 620, the frangible band 630 can separate from the body 610 along one of the first tear line 640A and second tear line 640B. In some embodiments, the cathlock body 610 retains its shape until the frangible band 630 is completely removed from the cathlock 600. Since the first tear line 640A does not extend to the first end 602, a portion of the frangible band 630 remains attached to body 610. A user can continue to pull on the tab 620, to remove the frangible band 630 and the cathlock body 610 in one movement.

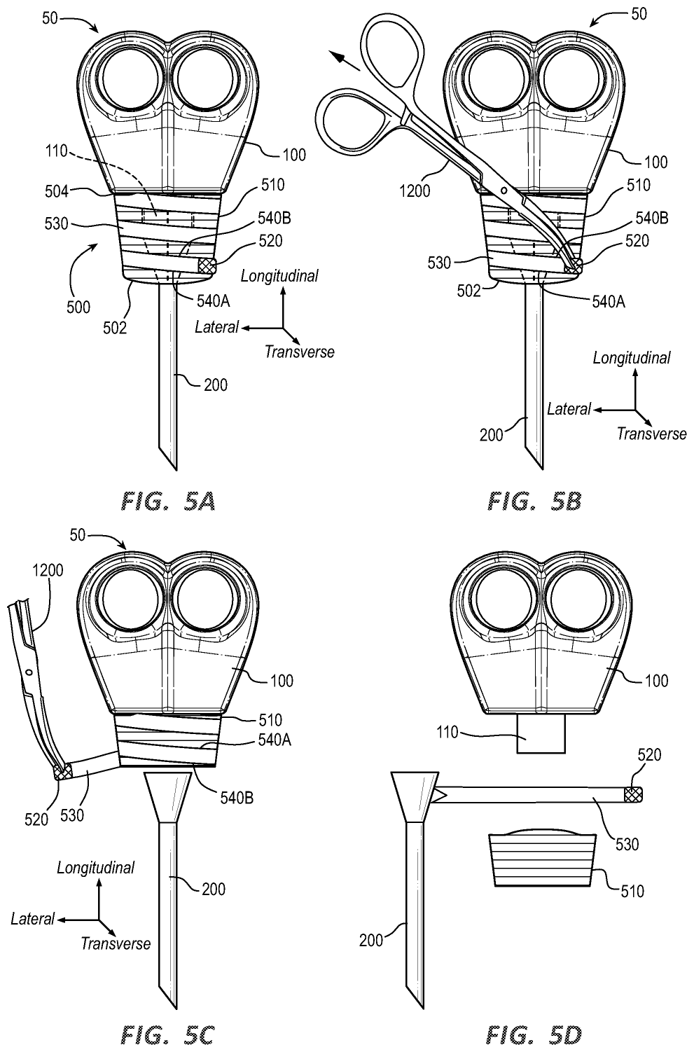

FIGS. 5A-5D illustrates an exemplary method of replacing a cathlock, for example the cathlock 500 of FIG. 2B, in accordance with some embodiments. FIG. 5A illustrates the catheter system 50 including a catheter 200, a peelable cathlock 300 and a port 100. The cathlock 500 can provide fluid communication between the catheter 200 and the port 100. The cathlock 500 can include a cathlock body 510, defining a first end 502 and a second end 504, a frangible band 530 defined by a first tear line 540A and a second tear line 540B, and a pull tab 520 coupled to the frangible band 530 at the first end 502 of the cathlock body 510. In an embodiment, the frangible band 630 can extend helically about the body 510 and adjacent portions of the helical frangible band 530 can include portions of the catheter body 510 interposed therebetween.

In an embodiment, one of the port 100 or the catheter 200 can be exchanged independently of each other by separating the catheter 200 from the port 100 by detaching the cathlock 300. As illustrated in FIG. 5B, a grasping medical device 1200, such as forceps, hemostats, or the like, can isolate and grasp the pull tab 520. The pull tab 520 can then be urged away from the catheter system 50, which can cause the frangible band 530 to separate from one of the cathlock body 510, or an adjacent portion of the frangible band 530 that form the cathlock body 510, along one of the first tear line 540A or the second tear line 540B.

In an embodiment, the cathlock 500 can be rotatably engaged with one of the port 100 or the catheter 200 about the longitudinal axis. As the frangible band 530 begins to separate from the cathlock body 510, the body 510 can rotate about the longitudinal axis allowing the cathlock body to unravel, as illustrated in FIG. 5C.

As noted, in an embodiment, the cathlock body 510 includes adjacent portions of the helical frangible band 530 interposed between the cathlock body 510. As such unraveling the frangible band 530 in this manner separates the frangible band 530 from the cathlock 500. The user can remove the cathlock 500 from the catheter system 50 allowing one of the port 100 or the catheter 200 to be exchanged. In some embodiments, when the pull tab 520 is coupled to the frangible band 530 at the first end 502 of the cathlock body 510, the unraveling of the frangible band 530 releases the catheter 200, while still being attached to the port 100. In some embodiments, when the pull tab 520 is coupled to the frangible band 530 at the second end 504 of the cathlock body 510, the unraveling of the frangible band 530 releases the port 100, while still being attached to the catheter 200.

FIG. 5D illustrates the complete unraveling of the frangible band 530 from the cathlock body 510, separating the catheter 200 from the port 100. The frangible band 530 can be removed and discarded, the user can then grasp and remove the cathlock body 510. One of the port 100 or the catheter 200 can then be exchanged.

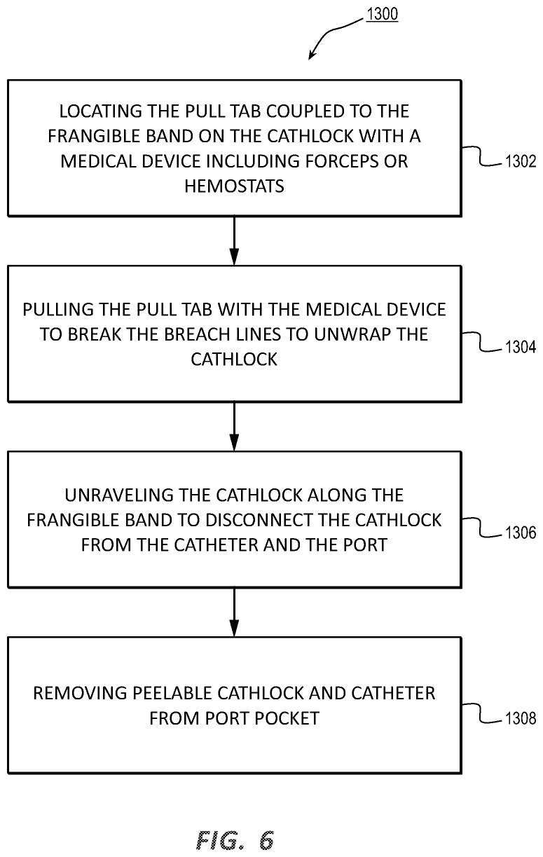

FIG. 6 illustrates a block diagram of the method 1300 of FIGS. 5A-5D. The first step (block 1302) includes locating the pull tab 520 coupled to the frangible band 530 on the cathlock 500 with a grasping medical device 1200 including forceps or hemostats, or the like. In some embodiments, the pull tab 520 can be located at a first end 502, a second end 504 of the cathlock body 510 or at a point therebetween. The second step (block 1304) includes pulling the pull tab 520 with the grasping medical device 1200 to separate the cathlock body 510 along at first tear line 540A and second tear line 540B to unwrap the cathlock 500. The third step (block 1306) includes separating the frangible band 530 to disconnect the cathlock 500 from the catheter 200 and the port 100. In some embodiments, the first tear line 540A and second tear line 540B separate in a clockwise or counterclockwise manner. In some embodiments, unraveling the cathlock 500 includes separating the frangible band 530 from the first end 502 to the second end 504 or from the second end 504 to the first end 502.

In some embodiments, unraveling the cathlock 500 includes unraveling only the frangible band 530 while the cathlock body 510 remains substantially stationary relative to the catheter 200 and the port 100. In some embodiments, unraveling the cathlock 500 includes the cathlock 500 rotating about the longitudinal axis as the frangible band 530 is unraveled helically. The fourth step (block 1308) includes removing the peelable cathlock 500 and one of the catheter 200 or the port 100 from the port pocket. In some embodiments, removing the peelable cathlock 500 includes removing the cathlock 500 as a single piece. In some embodiments, the removing the peelable cathlock 500 includes removing the cathlock 500 in two or more pieces.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A peelable cathlock, comprising:
a body defining a cylindrical shape and extending from a first end to a second end, the body defining a lumen providing fluid communication between the first end and the second end, the first end is configured to attach to a proximal end of a catheter, and the second end is configured to attach to a port stem; and
a tear line extending along a portion of the body and configured to allow the body to separate therealong when a force is applied.

2. The peelable cathlock according to claim 1, wherein the tear line includes one of a score line, groove, laser cut line, perforation, a thinned portion of a wall of the body.

3. The peelable cathlock according to claim 1, further including a frangible band extending along a portion of the body and defined by a first tear line and a second tear line.

4. The peelable cathlock according to claim 3, further including a pull tab coupled to one of the body or the frangible band, proximate one of the first end or the second end, and configured to allow a user to grasp the pull tab and urge the pull tab away from the body to separate the body along one of the first tear line or the second tear line.

5. The peelable cathlock according to claim 3, wherein the frangible band extends in a helical shape about a longitudinal axis of the body.

6. The peelable cathlock according to claim 5, wherein a first portion of the helical frangible band is disposed adjacent a second portion of the helical frangible band to form a portion of the body.

7. The peelable cathlock according to claim 3, wherein the frangible band extends parallel to a longitudinal axis of the body between the first end and the second end.

8. The peelable cathlock according to claim 3, wherein the first tear line extends from the first end to the second end and the second tear line extends along a portion of the body from one of the first end or the second end.

9. The peelable cathlock according to claim 3, wherein the frangible band extends along a portion of the body at an angle relative to longitudinal axis.

10. The peelable cathlock according to claim 1, wherein the cathlock includes one of a plastic, polymer, elastomer, polytetrafluoroethylene (PTFE), metal, or alloy material.

11. A method of removing a cathlock from a catheter system, comprising:
providing a cathlock including a body defining a substantially cylindrical shape and extending from a first end to a second end, and a tear line extending along a portion of the body, the cathlock providing fluid communication between a port and a catheter;
grasping a portion of the cathlock;
urging the portion of the cathlock away from the catheter system; and
separating the cathlock along the tear line.

12. The method according to claim 11, wherein the tear line includes one of a score line, groove, laser cut line, perforation, or a thinned portion of a wall of the body.

13. The method according to claim 11, further including a frangible band extending along a portion of the body and defined by a first tear line and a second tear line.

14. The method according to claim 13, wherein grasping a portion of the cathlock further includes grasping a pull tab coupled to one of the body or the frangible band, proximate one of the first end or the second end.

15. The method according to claim 13, wherein the frangible band extends in a helical shape about a longitudinal axis of the body.

16. The method according to claim 15, wherein a first portion of the helical frangible band is disposed adjacent a second portion of the helical frangible band to form a portion of the body.

17. The method according to claim 13, wherein the frangible band extends parallel to a longitudinal axis of the body between the first end and the second end.

18. The method according to claim 13, wherein the first tear line extends from the first end to the second end and the second tear line extends along a portion of the body from one of the first end or the second end.

19. The method according to claim 13, wherein the frangible band extends along a portion of the body at an angle relative to longitudinal axis.

20. The method according to claim 11, wherein the cathlock includes one of a plastic, polymer, elastomer, polytetrafluoroethylene (PTFE), metal, or alloy material.
